(19) United States
(12) Patent Application Publication
(10) Pub. No.: US 2026/0014554 A1
(43) Pub. Date: Jan. 15, 2026

(54) METHOD FOR REJUVENATING A CATALYST FROM A HYDROPROCESSING AND/OR HYDROCRACKING PROCESS

(71) Applicant: IFP ENERGIES NOUVELLES, Rueil-Malmaison (FR)

(72) Inventors: Elodie DEVERS, Rueil-Malmaison Cedex (FR); Sylvie LOPEZ

(73) Assignee: IFP ENERGIES NOUVELLES, Rueil-Malmaison (FR)

(21) Appl. No.: 18/994,343

(22) PCT Filed: Jun. 28, 2023
(86) PCT No.: PCT/EP2023/067562

§ 371 Date: Jan. 14, 2025

(30) Foreign Application Priority Data
Jul. 22, 2022 (FR) FR2207544

(51) Int. Cl. B01J 38/12; B01J 21/04; B01J 27/19; B01J 27/28; B01J 37/20; B01J 38/02; B01J 38/62; C07C 5/10

(52) U.S. Cl. CPC B01J 38/12; B01J 21/04; B01J 27/19; B01J 27/285; B01J 37/20; B01J 38/02; B01J 38/62; C07C 5/10; C07C 2523/883

(57) ABSTRACT

Process for the rejuvenation of a catalyst comprising a metal from group VIII, a metal from group VIb and an oxide support not comprising zeolite, comprising the following stages:
a) said catalyst is regenerated at a temperature of between 360° C. and less than 420° C. so as to obtain a regenerated catalyst comprising a certain content of carbon and sulfur and a proportion of crystalline phase determined by X-ray diffraction and characterized by a ratio of less than 0.6,
b) said regenerated catalyst is brought into contact with an aqueous solution consisting of water, of phosphoric acid and of an organic acid having each acidity constant pKₐ greater than 1.5,
c) drying is carried out at a temperature of less than 200° C.

### FIELD OF THE INVENTION

The invention relates to a process for the rejuvenation of a hydrotreating and/or hydrocracking catalyst and to the use of the rejuvenated catalyst in the field of hydrotreating and/or hydrocracking.

### PRIOR ART

Usually, a catalyst for the hydrotreating of hydrocarbon cuts has the aim of removing the sulfur-based or nitrogen-based compounds contained in them in order, for example, to make a petroleum product meet the required specifications (sulfur content, aromatics content, and the like) for a given application (motor vehicle fuel, gasoline or gas oil, domestic fuel oil, jet fuel).

Conventional hydrotreating catalysts generally comprise an oxide support and an active phase based on metals from groups VIb and Vill in their oxide forms and also phosphorus. The preparation of these catalysts generally comprises a stage of impregnation of the metals and the phosphorus on the support, followed by a drying operation and a calcination making it possible to obtain the active phase in their oxide forms. Before their use in a hydrotreating and/or hydrocracking reaction, these catalysts are generally subjected to a sulfidation in order to form the active entity.

The addition of an organic compound to hydrotreating catalysts in order to improve their activity has been recommended by a person skilled in the art, in particular for catalysts which have been prepared by impregnation followed by a drying operation without subsequent calcination. These catalysts are often referred to as “additive-impregnated dried catalysts”.

During its operation in a hydrotreating and/or hydrocracking process, the catalyst becomes deactivated by accumulation of coke and/or sulfur-based compounds or compounds containing other heteroelements at the surface of the catalyst. Beyond a certain period, its replacement is thus necessary.

In order to combat these disadvantages, the regeneration of catalysts for the hydrotreating of middle distillates or of spent residues is an economically and ecologically advantageous process because it makes it possible to use these catalysts again in industrial units rather than to landfill them or to recycle them (recovery of the metals). However, the regenerated catalysts are generally less active than the starting catalysts.

In order to overcome the shortfall in hydrodesulfurizing activity of the regenerated catalyst, it is possible to apply an additional “rejuvenation” treatment. The rejuvenation process consists in reimpregnating the regenerated catalyst with a solution containing metal precursors in the presence or absence of organic or inorganic additives. These “rejuvenation” processes are well known to a person skilled in the art in the field of middle distillates. Many patents, such as, for example, U.S. Pat. Nos. 7,906,447, 8,722,558, 7,956,000, 7,820,579, WO2005/070543, FR 2 972 648, US2017/036202, WO2001/02091, WO2001/02092 or also CN102463127, thus provide different methods for carrying out the rejuvenation of the catalysts for the hydrotreating of middle distillates.

The document U.S. Pat. No. 7,956,000 in particular describes a rejuvenation process which brings a catalyst comprising an oxide of metal from group VIb and an oxide of metal from group VIII into contact with an acid and an organic additive, the boiling point of which is of between 80° C. and 500° C. and a solubility in water of at least 5 grams per liter (20° C., atmospheric pressure), optionally followed by a drying operation under conditions such that at least 50% of the additive is maintained in the catalyst. The hydrotreating catalyst can be a fresh hydrotreating catalyst or a spent hydrotreating catalyst which has been regenerated.

The document FR 3 089 826 describes a rejuvenation process in which a catalyst comprising an oxide of metal from group VIb and an oxide of metal from group VIII is brought into contact with phosphoric acid and an organic acid having each acidity constant pKₐ greater than 1.5, followed by a drying operation at a temperature of less than 200° C. without subsequent calcination. The regeneration is carried out beforehand at a temperature between 300° C. and 500° C., preferably between 420° C. and 500° C. More particularly, the document FR 3 089 826 describes that the combination of phosphoric acid with an organic acid having each acidity constant pKₐ greater than 1.5 and preferably greater than 3.5, that is to say a not too strong organic acid, makes it possible to observe a synergistic effect at the level of the catalytic activity which is not foreseeable when phosphoric acid or organic acid is used alone.

The objective of the present invention is to provide an improvement to the rejuvenation process described in FR 3 089 826 by carrying out a controlled regeneration in a precise temperature range.

### Subject Matters of the Invention

The invention relates to a process for the rejuvenation of an at least partially spent catalyst resulting from a hydrotreating and/or hydrocracking process, said at least partially spent catalyst resulting from a fresh catalyst comprising at least one metal from group VIII, at least one metal from group VIb, an oxide support not comprising zeolite, and optionally phosphorus, said process comprises the following stages:
a) the at least partially spent catalyst is regenerated in an oxygen-containing gas stream at a temperature of between 360° C. and less than 420° C. so as to obtain a regenerated catalyst comprising a carbon content of between 0.1% and 0.5% by weight, a sulfur content of between 0.3% and 0.8% by weight and a proportion of crystalline phase resulting from at least one metal from group VIII and from at least one metal from group VIb determined by X-ray diffraction and characterized by a ratio of the surface area of the diffraction peak of the crystal at 26.6° 20 to the surface area of the peak characteristic of alumina at 45.7° 20 of less than 0.6,
b) then said regenerated catalyst is brought into contact with an aqueous solution consisting of water, of phosphoric acid and of an organic acid having each acidity constant pKₐ greater than 1.5,
c) a drying stage is carried out at a temperature of less than 200° C., without subsequently calcining it, so as to obtain a rejuvenated catalyst.

Generally, the regeneration is carried out in order to remove the coke and/or the sulfur-based compounds and/or other heteroelements which have accumulated in the catalyst during its use. The disappearance of the coke and of the other impurities by regeneration makes it possible to unblock the pores of the catalyst and thus to make the active phase again accessible to the feedstock. The higher the regeneration temperature, the more the content of coke and of other impurities is reduced.

However, regeneration at too high a temperature brings about the appearance of a crystalline phase resulting from at least one metal from group VIII and from at least one metal from group VIb (for example nickel molybdate NiMoO₄ and/or cobalt molybdate CoMoO₄) which is formed by sintering of the oxide precursors of the active phase which is composed of metals from group VIII and/or of metals from group VIb. The formation of such a crystalline phase is not desired because it results in catalytically non-active entities after activation by sulfidation.

The choice of the regeneration temperature is thus conflicting. On the one hand, the temperature must be high enough to remove the coke and/or the other impurities in order to free access to the active phase and, on the other hand, the temperature must not be too high in order to avoid the formation of the crystalline phase. The regeneration temperature conditions must thus be chosen in order to obtain a regenerated catalyst which contains between 0.1% and 0.5% by weight of carbon, between 0.3% and 0.8% by weight of sulfur, and a proportion of crystalline phase resulting from at least one metal from group VIII and from at least one metal from group VIb determined by X-ray diffraction and characterized by a ratio of the surface area of the diffraction peak of the crystal at 26.6° 2θ to the surface area of the peak characteristic of alumina at 45.7° 2θ of less than 0.6. The regeneration temperature conditions which make it possible to obtain such a solid are between 360° C. and less than 420° C.

The rejuvenation in the presence of two specific acids used on a regenerated catalyst which contains between 0.1% and 0.5% by weight of carbon, between 0.3% and 0.8% by weight of sulfur, and a proportion of crystalline phase resulting from at least one metal from group VIII and from at least one metal from group VIb characterized by said ratio of less than 0.6, with regeneration at a temperature of between 360° C. and less than 420° C., leads to a solid with a proportion of crystalline phase resulting from at least one metal from group VIII and from at least one metal from group VIb determined by X-ray diffraction and characterized by a ratio of the surface area of the diffraction peak of the crystal at 26.6° 2θ to the surface area of the peak characteristic of alumina at 45.7° 2θ of less than 0.4. Such a rejuvenation thus seems to make possible good dissolution of the crystalline phase and a dispersion of the metal phases in order to recover a dispersion close to the fresh catalyst and thus an activity close to the fresh catalyst, this being the case without it being necessary to add metals of the active phase.

This is because the applicant company has observed that the implementation of this rejuvenation process made it possible to obtain a hydrotreating and/or hydrocracking catalyst with improved catalytic performance qualities compared with the catalyst rejuvenated starting from a regenerated catalyst which would not meet the combined characteristics of contents of carbon, sulfur and crystalline phase.

By virtue of the controlled regeneration, the catalytic performance qualities are improved in such a way that the addition of metals during the rejuvenation is not necessary.

The enhancement in catalytic performance qualities can be observed on cobalt-or nickel-based catalysts but very particularly on nickel-based catalysts.

Typically, by virtue of the improvement in the activity, the temperature necessary to achieve a desired sulfur or nitrogen content (for example 10 ppm of sulfur in the case of a gas oil feedstock, in ULSD or Ultra Low Sulfur Diesel mode) is close to that of the fresh catalyst.

According to one alternative form, the temperature of stage a) is of between 380° C. and 410° C.

According to one alternative form, the proportion of crystalline phase resulting from at least one metal from group Vill and from at least one metal from group VIb determined by X-ray diffraction and characterized by a ratio of the surface area of the diffraction peak of the crystal at 26.6° 2θ to the surface area of the peak characteristic of alumina at 45.7° 2θ in stage a) is less than 0.50.

According to one alternative form, the organic acid used in stage b) is chosen from acetic acid, maleic acid, malic acid, malonic acid, gluconic acid, tartaric acid, citric acid, γ-ketovaleric acid, lactic acid, pyruvic acid, ascorbic acid or succinic acid.

According to one alternative form, the organic acid used in stage b) is an organic acid having each acidity constant pKₐ greater than 3.5.

According to one alternative form, the organic acid used in stage b) is chosen from gluconic acid, γ-ketovaleric acid, lactic acid, pyruvic acid, ascorbic acid or succinic acid.

According to one alternative form, the organic acid added per metal/metals from group VIb present in the regenerated catalyst molar ratio is of between 0.01 and 5 mol/mol.

According to one alternative form, the phosphorus added per metal from group VIb already present in the regenerated catalyst molar ratio is of between 0.01 and 5 mol/mol.

According to one alternative form, the fresh catalyst has a content of metal from group VIb of between 1% and 40% by weight of oxide of said metal from group VIb, with respect to the weight of the catalyst, and a total content of metal from group VIII of between 1% and 10% by weight of oxide of said metal from group VIII, with respect to the weight of the catalyst.

According to one alternative form, the fresh catalyst contains phosphorus, the total content of phosphorus being of between 0.1% and 20% by weight, expressed as P₂O₅, with respect to the total weight of the catalyst.

According to one alternative form, the oxide support not comprising zeolite is chosen from aluminas, silica, silica-aluminas or also titanium or magnesium oxides, used alone or as a mixture with alumina or silica-alumina.

According to one alternative form, the rejuvenated catalyst resulting from stage c) contains a proportion of crystalline phase resulting from at least one metal from group VIII and from at least one metal from group VIb determined by X-ray diffraction and characterized by a ratio of the surface area of the diffraction peak of the crystal at 26.6° 2θ to the surface area of the peak characteristic of alumina at 45.7° 2θ of less than 0.4.

According to one alternative form, the regeneration stage a) is preceded by a deoiling stage which comprises bringing an at least partially spent catalyst resulting from a hydrotreating and/or hydrocracking process into contact with a stream of inert gas at a temperature of between 300° C. and 400° C.

According to one alternative form, the rejuvenated catalyst is subjected to a sulfidation stage after stage c).

The invention also relates to the use of the rejuvenated catalyst prepared according to the process of the invention in a process for the hydrotreating and/or hydrocracking of hydrocarbon cuts.

According to the present invention, the expressions “of between . . . and . . . ” and “between . . . and . . . ” are equivalent and mean that the limiting values of the interval are included in the described range of values. If such were not the case and if the limiting values were not included in the range described, such a clarification will be introduced by the present invention.

Within the meaning of the present invention, the various parameter ranges for a given stage, such as the pressure ranges and the temperature ranges, can be used alone or in combination. For example, within the meaning of the present invention, a range of preferred pressure values can be combined with a range of more preferred temperature values.

Subsequently, particular and/or preferred embodiments of the invention may be described. They can be implemented separately or combined together, without limitation of combination when this is technically feasible.

Subsequently, the groups of chemical elements are given according to the CAS classification (CRC Handbook of Chemistry and Physics, published by CRC Press, editor-in-chief D. R. Lide, 81^{st} edition, 2000-2001). For example, group VIII (or VIIIb) according to the CAS classification corresponds to the metals of columns 8, 9 and 10 according to the new IUPAC classification.

According to the present invention, the pressures are absolute pressures, also denoted abs., and are given in MPa absolute (or MPa abs.), unless otherwise indicated.

The content of metals is measured by X-ray fluorescence.

Hydrotreating is understood to mean reactions encompassing in particular hydrodesulfurization (HDS), hydrodenitrogenation (HDN) and hydrogenation of aromatics (HOA).

### DESCRIPTION OF THE INVENTION

The rejuvenated catalyst obtained by the process according to the invention results from an at least partially spent catalyst, itself resulting from a fresh catalyst, used in a process for the hydrotreating and/or hydrocracking of hydrocarbon cuts for a certain period of time and which exhibits a significantly lower activity than the fresh catalyst, which necessitates its replacement.

### Fresh Catalyst

The fresh catalyst used in a process for the hydrotreating and/or hydrocracking of hydrocarbon cuts is known to a person skilled in the art. It comprises at least one metal from group VIII, at least one metal from group VIb, an oxide support not comprising zeolite, and optionally phosphorus and/or an organic compound as described below.

The metal from group VIb present in the active phase of the fresh catalyst is preferentially chosen from molybdenum and tungsten. The metal from group Vill present in the active phase of the fresh catalyst is preferentially chosen from cobalt, nickel and the mixture of these two elements. The active phase of the fresh catalyst is preferably chosen from the group formed by the combination of the elements nickel-molybdenum, cobalt-molybdenum, nickel-tungsten, nickel-molybdenum-tungsten and nickel-cobalt-molybdenum and very preferably the active phase consists of cobalt and molybdenum, nickel and molybdenum, nickel and tungsten or a nickel-molybdenum-tungsten combination. In a particularly preferred way, the active phase consists of nickel and of molybdenum.

The content of metal from group VIII is of between 1% and 10% by weight, preferably between 1.5% and 9% by weight and more preferably between 2% and 8% by weight, expressed as oxide of metal from group VIII, with respect to the total weight of the fresh catalyst.

The content of metal from group VIb is of between 1% and 40% by weight, preferably between 1% and 35% by weight and more preferably between 2% and 30% by weight, expressed as oxide of metal from group VIb, with respect to the total weight of the fresh catalyst.

The metal from group VIII to metal from group VIb molar ratio of the fresh catalyst is generally of between 0.1 and 0.8, preferably of between 0.15 and 0.6.

Optionally, the fresh catalyst can additionally exhibit a phosphorus content generally of between 0.1% and 20% by weight of P₂O₅, with respect to the total weight of fresh catalyst, preferably of between 0.2% and 15% by weight of P₂O₅, very preferably of between 0.3% and 11% by weight of P₂O₅. For example, the phosphorus present in the fresh catalyst is combined with the metal from group VIb and optionally also with the metal from group VIII in the form of heteropolyanions.

Furthermore, the phosphorus/(metal from group VIb) molar ratio is generally of between 0.08 and 1, preferably of between 0.1 and 0.9 and very preferably of between 0.15 and 0.8.

The oxide support not comprising zeolite of the fresh catalyst is usually a porous solid chosen from the group consisting of: aluminas, silica, silica-aluminas or also titanium and magnesium oxides, used alone or as a mixture with alumina or silica-alumina. Preferably, the oxide support not comprising zeolite is a support based on alumina or on silica or on silica-alumina.

When the oxide support not comprising zeolite is based on alumina, it contains more than 50% by weight of alumina, with respect to the total weight of the support, and, in general, it contains only alumina or silica-alumina as defined below.

Preferably, the oxide support not comprising zeolite comprises alumina and preferably extruded alumina. Preferably, the alumina is gamma alumina.

The alumina support advantageously exhibits a total pore volume of between 0.1 and 1.5 cm³ g⁻¹, preferably between 0.4 and 1.1 cm³·g⁻¹. The total pore volume is measured by mercury porosimetry according to the standard ASTM D4284 with a wetting angle of 140°, as described in the work by Rouquerol F., Rouquerol J. and Sing K., “Adsorption by Powders & Porous Solids: Principle, Methodology and Applications”, Academic Press, 1999, for example by means of an Autopore III™ model machine of the Micromeritics™ brand.

The specific surface of the alumina support is advantageously of between 5 and 400 m²·g⁻¹, preferably between 10 and 350 m²·g⁻¹, more preferably between 40 and 350 m²·g⁻¹. The specific surface is determined in the present invention by the BET method according to the standard ASTM D3663, which method is described in the same work mentioned above.

In another preferred case, the oxide support is a silica-alumina containing at least 50% by weight of alumina, with respect to the total weight of the support. The silica content in the support is at most 50% by weight, with respect to the total weight of the support, generally less than or equal to 45% by weight, preferably less than or equal to 40%.

Sources of silicon are well known to a person skilled in the art. Mention may be made, by way of example, of silicic acid, silica in the powder form or in the colloidal form (silica sol), or tetraethyl orthosilicate Si(OEt)₄.

When the support for said catalyst is based on silica, it contains more than 50% by weight of silica, with respect to the total weight of the support, and, generally, it contains only silica.

According to a particularly preferred alternative form, the oxide support consists of alumina, silica or silica-alumina.

The support is advantageously provided in the form of beads, extrudates, pellets or irregular and non-spherical agglomerates, the specific shape of which can result from a crushing stage.

The fresh catalyst can also additionally comprise at least one organic compound containing oxygen and/or nitrogen and/or sulfur before sulfidation. Such additives are known. Generally, the organic compound is chosen from a compound comprising one or more chemical functions chosen from a carboxylic, alcohol, thiol, thioether, sulfone, sulfoxide, ether, aldehyde, ketone, ester, carbonate, amine, nitrile, imide, oxime, urea and amide function or also compounds including a furan ring or also sugars.

The oxygen-containing organic compound can be one or more chosen from compounds comprising one or more chemical functions chosen from a carboxylic, alcohol, ether, aldehyde, ketone, ester or carbonate function or also compounds including a furan ring or also sugars. By way of example, the oxygen-containing organic compound can be one or more chosen from the group consisting of ethylene glycol, diethylene glycol, triethylene glycol, a polyethylene glycol (with a molecular weight of between 200 and 1500 g/mol), propylene glycol, 2-butoxyethanol, 2-(2-butoxyethoxy) ethanol, 2-(2-methoxyethoxy) ethanol, triethylene glycol dimethyl ether, glycerol, acetophenone, 2,4-pentanedione, pentanone, acetic acid, maleic acid, malic acid, malonic acid, oxalic acid, gluconic acid, tartaric acid, citric acid, γ-ketovaleric acid, a di(C₁-C₄ alkyl) succinate and more particularly dimethyl succinate, methyl acetoacetate, ethyl acetoacetate, 2-methoxyethyl 3-oxobutanoate, 2-methacryloyloxyethyl 3-oxobutanoate, dibenzofuran, a crown ether, orthophthalic acid, glucose, fructose, sucrose, sorbitol, xylitol, γ-valerolactone, 2-acetylbutyrolactone, propylene carbonate, 2-furaldehyde (also known as furfural), 5-hydroxymethylfurfural (also known under the name 5-(hydroxymethyl)-2-furaldehyde or 5-HMF), 2-acetylfuran, 5-methyl-2-furaldehyde, methyl 2-furoate, furfuryl alcohol (also known under the name furfuranol), furfuryl acetate, ascorbic acid, butyl lactate, butyl butyryllactate, ethyl 3-hydroxybutanoate, ethyl 3-ethoxypropanoate, methyl 3-methoxypropanoate, 2-ethoxyethyl acetate, 2-butoxyethyl acetate, 2-hydroxyethyl acrylate, 2-hydroxyethyl methacrylate and 5-methyl-2 (3H)-furanone.

The nitrogen-containing organic compound can be one or more chosen from compounds comprising one or more chemical functions chosen from an amine or nitrile function. By way of example, the nitrogen-containing organic compound can be one or more chosen from the group consisting of ethylenediamine, diethylenetriamine, hexamethylenediamine, triethylenetetramine, tetraethylenepentamine, pentaethylenehexamine, acetonitrile, octylamine, guanidine and a carbazole.

The organic compound containing oxygen and nitrogen can be one or more chosen from compounds comprising one or more chemical functions chosen from a carboxylic acid, alcohol, ether, aldehyde, ketone, ester, carbonate, amine, nitrile, imide, amide, urea or oxime function.

By way of example, the organic compound containing oxygen and nitrogen can be one or more chosen from the group consisting of 1,2-cyclohexanediaminetetraacetic acid, monoethanolamine (MEA), 1-methyl-2-pyrrolidinone, dimethylformamide, ethylenediaminetetraacetic acid (EDTA), alanine, glycine, nitrilotriacetic acid (NTA), N-(2-hydroxyethyl)ethylenediamine-N,N′, N′-triacetic acid (HEDTA), diethylenetriaminepentaacetic acid (DTPA), tetramethylurea, glutamic acid, dimethylglyoxime, bicine, tricine, 2-methoxyethyl cyanoacetate, 1-ethyl-2-pyrrolidinone, 1-vinyl-2-pyrrolidinone, 1,3-dimethyl-2-imidazolidinone, 1-(2-hydroxyethyl)-2-pyrrolidinone, 1-(2-hydroxyethyl)-2,5-pyrrolidinedione, 1-methyl-2-piperidinone, 1-acetyl-2-azepanone, 1-vinyl-2-azepanone and 4-aminobutanoic acid.

The sulfur-containing organic compound can be one or more chosen from compounds comprising one or more chemical functions chosen from a thiol, thioether, sulfone or sulfoxide function. By way of example, the sulfur-containing organic compound can be one or more chosen from the group consisting of thioglycolic acid, 2,2′-thiodiethanol, 2-hydroxy-4-methylthiobutanoic acid, a sulfone derivative of a benzothiophene or a sulfoxide derivative of a benzothiophene, methyl 3-(methylthio) propanoate and ethyl 3-(methylthio) propanoate.

Preferably, the organic compound contains oxygen and preferably it contains only oxygen as heteroatom. Preferably, it is chosen from γ-valerolactone, 2-acetylbutyrolactone, triethylene glycol, diethylene glycol, ethylene glycol, ethylenediaminetetraacetic acid (EDTA), maleic acid, malonic acid, citric acid, gluconic acid, dimethyl succinate, glucose, fructose, sucrose, sorbitol, xylitol, γ-ketovaleric acid, dimethylformamide, 1-methyl-2-pyrrolidinone, propylene carbonate, 2-methoxyethyl 3-oxobutanoate, bicine, tricine, 2-furaldehyde (also known under the name furfural), 5-hydroxymethylfurfural (also known under the name 5-(hydroxymethyl)-2-furaldehyde or 5-HMF), 2-acetylfuran, 5-methyl-2-furaldehyde, ascorbic acid, butyl lactate, ethyl 3-hydroxybutanoate, ethyl 3-ethoxypropanoate, 2-ethoxyethyl acetate, 2-butoxyethyl acetate, 2-hydroxyethyl acrylate, 1-vinyl-2-pyrrolidinone, 1,3-dimethyl-2-imidazolidinone, 1-(2-hydroxyethyl)-2-pyrrolidinone, 1-(2-hydroxyethyl)-2,5-pyrrolidinedione, 5-methyl-2 (3H)-furanone, 1-methyl-2-piperidinone and 4-aminobutanoic acid.

The content of organic compound(s) containing oxygen and/or nitrogen and/or sulfur on the fresh catalyst is of between 1% and 30% by weight, preferably between 1.5% and 25% by weight and more preferably between 2% and 20% by weight, with respect to the total weight of the fresh catalyst.

The preparation of the fresh catalyst is known and generally comprises a stage of impregnation of the metals from group VIII and from group VIb and optionally of phosphorus and/or of the organic compound on the oxide support not comprising zeolite, followed by a drying operation, then by an optional calcination making it possible to obtain the active phase in their oxide forms. Before its use in a process for the hydrotreating and/or hydrocracking of hydrocarbon cuts, the fresh catalyst is generally subjected to a sulfidation in order to form the active entity as described below.

The impregnation stage of the preparation of the fresh catalyst can be carried out either by slurry impregnation, or by impregnation in excess, or by dry impregnation, or by all other means known to a person skilled in the art.

Use may be made, by way of example, among the sources of molybdenum, of the oxides and hydroxides, molybdic acids and their salts, in particular the ammonium salts, such as ammonium molybdate or ammonium heptamolybdate, phosphomolybdic acid (H₃PMo₁₂O₄₀) and their salts, and optionally silicomolybdic acid (H₄SiMo₁₂O₄₀) and its salts. The sources of molybdenum can also be any heteropolycompound of Keggin, lacunary Keggin, substituted Keggin, Dawson, Anderson or Strandberg type, for example. Use is preferably made of molybdenum trioxide and the heteropolycompounds of Keggin, lacunary Keggin, substituted Keggin and Strandberg type.

The tungsten precursors which can be used are also well known to a person skilled in the art. For example, use may be made, among the sources of tungsten, of the oxides and hydroxides, tungstic acids and their salts, in particular the ammonium salts, such as ammonium tungstate or ammonium metatungstate, phosphotungstic acid and their salts, and optionally silicotungstic acid (H₄SiW₁₂O₄₀) and its salts. The sources of tungsten can also be any heteropolycompound of Keggin, lacunary Keggin, substituted Keggin or Dawson type, for example. Use is preferably made of the oxides and the ammonium salts, such as ammonium metatungstate, or the heteropolyanions of Keggin, lacunary Keggin or substituted Keggin type.

The cobalt precursors which can be used are advantageously chosen from the oxides, hydroxides, hydroxycarbonates, carbonates and nitrates, for example. Use is preferably made of cobalt hydroxide and cobalt carbonate.

The nickel precursors which can be used are advantageously chosen from the oxides, hydroxides, hydroxycarbonates, carbonates and nitrates, for example. Use is preferably made of nickel hydroxide and nickel hydroxycarbonate.

The preferred phosphorus precursor is orthophosphoric acid H₃PO₄ but its salts and esters, such as ammonium phosphates, are also suitable. The phosphorus can also be introduced at the same time as the element(s) from group VIb in the form of Keggin, lacunary Keggin, substituted Keggin or Strandberg-type heteropolyanions.

The impregnation stage comprises several embodiments. They are distinguished in particular by the moment at which the organic compound, when it is present, is introduced, which introduction can be carried out at the same time as the impregnation of the compound comprising a metal from group VIb (coimpregnation), or after (postimpregnation), or before (preimpregnation). In addition, it is possible to combine the embodiments.

Advantageously, after each impregnation stage, whether this is a stage of impregnation with the metals and optionally with the phosphorus or with the organic compound, the impregnated support is left to mature.

Any maturation stage is advantageously carried out at atmospheric pressure, in a water-saturated atmosphere and at a temperature of between 17° C. and 50° C., and preferably at ambient temperature. Generally, a maturation time of between ten minutes and forty-eight hours and preferably of between thirty minutes and six hours is sufficient.

The impregnation solution can comprise any polar solvent known to a person skilled in the art. Said polar solvent used is advantageously chosen from the group formed by methanol, ethanol, water, phenol and cyclohexanol, taken alone or as a mixture. Said polar solvent can also advantageously be chosen from the group formed by propylene carbonate, DMSO (dimethyl sulfoxide), N-methylpyrrolidone (NMP) and sulfolane, taken alone or as a mixture. Preferably, a polar protic solvent is used. A list of the common polar solvents and also their dielectric constants can be found in the book Solvents and Solvent Effects in Organic Chemistry, C. Reichardt, Wiley-VCH, 3rd edition, 2003, pages 472-474. Very preferably, the solvent used is water or ethanol and particularly preferably the solvent is water. In one possible embodiment, the solvent can be absent from the impregnation solution.

After the impregnation stage and an optional maturation stage, the catalyst is subjected to a drying stage at a temperature of less than 200° C., advantageously of between 50° C. and 180° C., preferably between 70° C. and 150° C., very preferably between 75° C. and 130° C., without a subsequent calcination stage. The drying stage is preferentially carried out under an inert atmosphere or under an oxygen-containing atmosphere.

According to an alternative form of the invention, which is preferred when an organic compound is present, the fresh catalyst has not undergone calcination during its preparation, that is to say that the impregnated catalytic precursor has not been subjected to a stage of heat treatment at a temperature of greater than 200° C. under an inert atmosphere or under an oxygen-containing atmosphere, in the presence or absence of water.

According to another alternative form of the invention, the fresh catalyst has undergone a calcination stage during its preparation, that is to say that the impregnated catalytic precursor has been subjected to a stage of heat treatment at a temperature of between 200° C. and 1000° C. and preferably between 250° C. and 750° C., for a period of time typically of between 15 minutes and 10 hours, under an inert atmosphere or under an oxygen-containing atmosphere, in the presence or absence of water.

During the process for the hydrotreating and/or hydrocracking of hydrocarbon cuts, coke and sulfur as well as other contaminants resulting from the feedstock, such as silicon, arsenic and metals, are formed and/or deposited on the catalyst and transform the fresh catalyst into an at least partially spent catalyst.

An at least partially spent catalyst is understood to mean a catalyst which exits from a hydrotreating process carried out under the conditions as described below and which has not undergone a heat treatment under a gas containing air or oxygen at a temperature of greater than 200° C. (also often known as regeneration stage). It may have undergone a deoiling.

The at least partially spent catalyst is composed of the oxide support not comprising zeolite and of the active phase formed of at least one metal from group VIb and of at least one metal from group VIII and optionally of the phosphorus from the fresh catalyst, as well as carbon, sulfur and optionally other contaminants resulting from the feedstock, such as silicon, arsenic and metals.

The contents of metal from group VIb, of metal from group VIII and of phosphorus in the fresh, at least partially spent, regenerated or rejuvenated catalyst are expressed as oxides after correction for the loss on ignition of the catalyst sample in a muffle furnace at 550° C. for two hours. The loss on ignition is due to the loss of moisture, carbon, sulfur and/or other contaminants. It is determined according to ASTM D7348.

The contents of metal from group VIb, of metal from group VIII and optionally of phosphorus in the at least partially spent catalyst are substantially identical to the contents of the fresh catalyst from which it results. The term “substantially identical” is understood to mean that each of the metal elements mentioned is present in the same proportions as in the initial fresh catalyst to within 5% relative.

It should be noted that the term “coke” or “carbon” in the present patent application denotes a substance based on hydrocarbons which is deposited on the surface of the at least partially spent hydrotreating catalyst during its use, which is highly cyclized and condensed and which has an appearance similar to graphite.

The at least partially spent catalyst contains in particular carbon at a content generally of greater than or equal to 2% by weight, preferably of between 2% and 25% by weight and more preferably still of between 4% and 16% by weight, with respect to the total weight of the at least partially spent catalyst.

The at least partially spent catalyst contains in particular sulfur at a content generally of greater than or equal to 2% by weight, preferably of between 2% and 25% by weight and more preferably still of between 4% and 16% by weight, with respect to the total weight of the at least partially spent catalyst.

### Regeneration (Stage a))

The process for the rejuvenation according to the invention of the at least partially spent catalyst comprises a stage of removal of the coke and the sulfur (regeneration stage). This is because, according to stage a) of the process according to the invention, the at least partially spent catalyst is regenerated in an oxygen-containing gas stream at a temperature of between 360° C. and less than 420° C. so as to obtain a regenerated catalyst comprising a carbon content of between 0.1% and 0.5% by weight, a sulfur content of between 0.3% and 0.8% by weight and a proportion of crystalline phase resulting from at least one metal from group VIII and from at least one metal from group VIb determined by X-ray diffraction and characterized by a ratio of the surface area of the diffraction peak of the crystal at 26.6° 2θ to the surface area of the peak characteristic of alumina at 45.7° 2θ of less than 0.6.

Even if this is possible, the regeneration is preferably not carried out by keeping the laden catalyst in the hydrotreating reactor (in situ regeneration). Preferably, the at least partially spent catalyst is thus extracted from the reactor and sent to a regeneration plant in order to carry out the regeneration in said plant (ex situ regeneration).

The regeneration stage a) is preferably preceded by a deoiling stage. The deoiling stage generally comprises bringing the at least partially spent catalyst into contact with a stream of inert gas (that is to say essentially devoid of oxygen), for example in a nitrogen atmosphere or the like, at a temperature of between 300° C. and 400° C., preferably of between 300° C. and 350° C. The inert gas flow rate in terms of flow rate per unit volume of the catalyst is from 5 to 150 SI.I⁻¹.h⁻¹ for 3 to 7 hours.

In an alternative form, the deoiling stage can be carried out by light hydrocarbons, by steam treatment or any other analogous process.

The deoiling stage makes it possible to remove the soluble hydrocarbons which might prove to be dangerous in the regeneration stage because they present risks of flammability under an oxidizing atmosphere.

The regeneration stage a) is generally carried out in a stream of oxygen-containing gas, generally air. The water content is generally of between 0% and 50% by weight. The gas flow rate in terms of flow rate per unit volume of the at least partially spent catalyst is preferably from 20 to 2000 SI.I⁻¹.h⁻¹, more preferably from 30 to 1000 SI.I⁻¹.h⁻¹ and particularly preferably from 40 to 500 SI.I⁻¹.h⁻¹. The duration of the regeneration is preferably 2 hours or more, more preferably 2.5 hours or more and particularly preferably 3 hours or more. The regeneration of the at least partially spent catalyst is generally carried out at a temperature of between 360° C. and less than 420° C., preferably of between 360° C. and 415° C., in a preferred way of between 360° C. and 410° C. or also of between 380° C. and 410° C.

The regeneration stage a) can be carried out, for example, in a traversed bed, in a swept bed or in a static atmosphere. For example, the oven used can be a rotating rotary oven or a vertical oven comprising radial traversed layers or also a band oven.

This is because it is important for the temperature to be high enough to remove the coke and/or the other impurities in order to free access to the active phase and, at the same time, it must not be too high in order to avoid the formation of the crystalline phase.

The regenerated catalyst is composed of the oxide support not comprising zeolite and of the active phase formed of at least one metal from group VIb and of at least one metal from group VIII and optionally of phosphorus from the fresh catalyst. Following the regeneration, the hydrogenating function comprising the metals from group VIb and from group VIII of the regenerated catalyst is in an oxide form.

The contents of metal from group VIb, of metal from group VIII and optionally of phosphorus in the regenerated catalyst are substantially identical to the contents of the at least partially spent catalyst and to the contents of the fresh catalyst from which it results. The term “substantially identical” is understood to mean that each of the metal elements mentioned is present in the same proportions as in the initial fresh catalyst to within 5% relative.

The regenerated catalyst is characterized by a specific surface of between 5 and 400 m²/g, preferably of between 10 and 350 m²/g, preferably of between 40 and 350 m²/g, very preferably of between 150 and 340 m²/g.

The pore volume of the regenerated catalyst is generally of between 0.1 cm³/g and 1.5 cm³/g, preferably of between 0.3 cm³/g and 1.1 cm³/g.

The regenerated catalyst obtained in the regeneration stage a) contains residual carbon at a content of between 0.1% and 0.5% by weight, with respect to the total weight of the regenerated catalyst, preferably of between 0.1% and 0.49% by weight, with respect to the total weight of the regenerated catalyst, preferentially of between 0.1% and 0.45% by weight and particularly preferably between 0.1% and 0.4% by weight.

It should be noted that the term “residual carbon” in the present patent application means carbon (coke) remaining in the regenerated catalyst after regeneration of the spent hydrotreating catalyst. This residual carbon content in the regenerated hydrotreating catalyst is measured by elemental analysis according to the standard ASTM D5373.

The regenerated catalyst can contain residual sulfur at a content of between 0.3% and 0.8% by weight, with respect to the total weight of the regenerated catalyst, preferably of between 0.3% and 0.75% by weight, with respect to the total weight of the regenerated catalyst, preferentially of between 0.4% and 0.75% by weight and particularly preferably between 0.4% and 0.7% by weight. This residual sulfur content in the regenerated hydrotreating catalyst is measured by elemental analysis according to ASTM D5373.

It is in fact surprising that a regeneration at a relatively low regeneration temperature makes it possible to obtain a regenerated catalyst containing only very little sulfur and/or residual carbon. A relatively low sulfur and/or residual carbon content is important in order to free access to the active phase.

During the regeneration stage, a part of the active phase can form a crystalline phase resulting from at least one metal from group VIII and from at least one metal from group VIb. The crystalline phase can be a single crystalline compound or a mixture of different crystalline compounds. According to the metal composition of the catalysts, different crystalline compounds can be formed, for example nickel molybdate NiMoO₄, cobalt molybdate CoMoO₄, nickel tungstate NiWO₄ or cobalt tungstate CoWO₄; mixtures of these or mixed metal crystals can also be formed.

The formation of such a crystalline phase is not desired because it results in catalytically non-active entities after activation by sulfidation and thus exhibits a loss of active phase.

The proportion of crystalline phase resulting from at least one metal from group VIII and from at least one metal from group VIb is characterized by a ratio of the surface area of the diffraction peak of the crystal at 26.6° 2θ to the surface area of the peak characteristic of alumina at 45.7° 2θ of less than 0.6, preferably of less than 0.55, in a preferred way of less than 0.5. The regenerated catalyst may also not contain crystalline phase.

The content of crystalline phase is measured by X-ray diffraction (XRD). The diffraction diagram is obtained by means of a diffractometer using the conventional powder method with the Kα₁ line of copper (λ=1.5406 Å). From the position of the diffraction peaks represented by the angle 20, the interplanar distances dₕₖᵢ characteristic of a crystalline phase and which make possible its identification based on the existing databases (COD Crystallography Open Database, ICDD International Center for Diffraction Data) are calculated by the Bragg relationship. The measurement error Δ(dₕₖᵢ) with regard to dₕₖᵢ is calculated by virtue of Bragg's law as a function of the absolute error Δ(2θ) assigned to the measurement of 2θ. The absolute error Δ(2θ) is ±0.5°. The relative area Aᵣₑₗ assigned to each dₕₖᵢ value is measured by integration of the corresponding diffraction peak after subtraction of the baseline. Software for integration and subtraction of the baseline is known and conventionally used by a person skilled in the art.

The proportion of the crystalline phase is evaluated relative to the signal of alumina from the X-ray diffractogram by evaluating the ratio of the surface area of the diffraction peak of the crystal, for example nickel molybdate (26.6° 2θ) or cobalt molybdate (also 26.6° 2θ) to the surface area of a peak characteristic of alumina (for example, that at 45.7° 2θ for γ-alumina), the surface areas having been calculated after subtraction of the baseline of the diffractogram.

Optionally, the regenerated catalyst can additionally exhibit a low content of contaminants resulting from the feedstock treated by the fresh catalyst from which it originates, such as silicon, arsenic and metals, such as nickel, vanadium or iron.

Preferably, the content of silicon (besides that possibly present on the fresh catalyst) is less than 2% by weight and very preferably less than 1% by weight, with respect to the total weight of the regenerated catalyst.

Preferably, the arsenic content is less than 2000 ppm by weight and very preferably less than 1000 ppm by weight, with respect to the total weight of the regenerated catalyst.

Preferably, the content for each of the metals, nickel, vanadium or iron, is less than 1% by weight and very preferably less than 5000 ppm by weight, with respect to the total weight of the regenerated catalyst.

### Rejuvenation (Stage b))

The rejuvenation process according to the invention comprises, after the regeneration stage a), a stage b) according to which said regenerated catalyst is brought into contact with an aqueous solution consisting of water, of phosphoric acid and of an organic acid having each acidity constant pKₐ greater than 1.5, preferably greater than 3.5.

The organic acid can contain one or more carboxylic functions, each acidity constant being greater than 1.5 and preferably greater than 3.0 and particularly preferably greater than 3.5. The acidity constant is measured at 25° C. in water. The organic acid can contain, in addition to the carboxylic function(s), other chemical functions of alcohol, ether, aldehyde, ketone or ester type.

The organic acid is preferably chosen from acetic acid, maleic acid, malic acid, malonic acid, gluconic acid, tartaric acid, citric acid, γ-ketovaleric acid, lactic acid, pyruvic acid, ascorbic acid or also succinic acid and preferably the organic acid is chosen from citric acid, acetic acid, gluconic acid, γ-ketovaleric acid, lactic acid, ascorbic acid and succinic acid.

Particularly preferably, the acid is an organic acid having each acidity constant pKₐ greater than 3.5. Preferably, the organic acid is chosen from gluconic acid, γ-ketovaleric acid, lactic acid, ascorbic acid or succinic acid.

These acids have the following acidity constants:

$$\begin{matrix}acetic acid & {pK}_{a} = 4.76 & & \\ maleic acid & {pK}_{a 1} = 1.89 & {pK}_{a 2} = 6.23 & \\ malic acid & {pK}_{a 1} = 3 \text{.46} & {pK}_{a 2} = 5.10 & \\ malonic acid & {pK}_{a 1} = 2 \text{.85} & {pK}_{a 2} = 5.70 & \\ gluconic acid & {pK}_{a} = 3.86 & & \\ tartaric acid & {pK}_{a 1} = 2 \text{.50} & {pK}_{a 2} = 4.20 & \\ citric acid & {pK}_{a 1} = 3 \text{.13} & {pK}_{a 2} = 4 \text{.76} & {pK}_{a 3} = 6.4 \\ γ - ketovaleric acid & {pK}_{a 1} = 4.64 & & \\ lactic acid & {pK}_{a} = 3.86 & & \\ pyruvic acid & {pK}_{a} = 2.49 & & \\ ascorbic acid & {pK}_{a 1} = 4 \text{.10} & {pK}_{a 2} = 11.8 & \\ succinic acid & {pK}_{a 1} = 4 \text{.21} & {pK}_{a 2} = 5.64 . & \end{matrix}$$

The organic acid is advantageously introduced into the aqueous impregnation solution in an amount corresponding:
to a molar ratio of organic acid added per metal/metals from group VIb present in the regenerated catalyst of between 0.01 and 5 mol/mol, preferably of between 0.05 and 3 mol/mol, in a preferred way of between 0.05 and 2 mol/mol and very preferably of between 0.1 and 1.5 mol/mol, and
to a molar ratio of organic acid added per metal/metals from group VIII present in the regenerated catalyst of between 0.02 and 17 mol/mol, preferably of between 0.1 and 10 mol/mol, in a preferred way of between 0.15 and 5 mol/mol and very preferably of between 0.2 and 3.5 mol/mol.

When several organic acids are present, the different molar ratios apply for each of the organic acids present.

With regard to the phosphoric acid, this is advantageously introduced into the aqueous impregnation solution in an amount corresponding to a molar ratio of phosphorus added per metal from group VIb already present in the regenerated catalyst of between 0.01 and 5 mol/mol, preferably of between 0.05 and 3 mol/mol, in a preferred way of between 0.05 and 2 mol/mol and very preferably of between 0.1 and 1.5 mol/mol.

Stage b) of bringing said into contact can be carried out by slurry impregnation, or by impregnation in excess, or by dry impregnation, or by all other means known to a person skilled in the art.

Equilibrium (or excess) impregnation consists in immersing the support or the catalyst in a volume of solution (often considerably) greater than the pore volume of the support or of the catalyst while keeping the system stirred in order to improve the exchanges between the solution and the support or catalyst. An equilibrium is finally reached after diffusion of the different entities into the pores of the support or catalyst. Control of the amount of elements deposited is provided by the prior measurement of an adsorption isotherm which relates the concentration of the elements to be deposited contained in the solution to the amount of the elements deposited on the solid in equilibrium with this solution.

Dry impregnation consists, for its part, in introducing a volume of impregnation solution equal to the pore volume of the support or of the catalyst. Dry impregnation makes it possible to deposit, on a given support or catalyst, all of the components contained in the impregnation solution.

Stage b) can advantageously be carried out by one or more excess impregnations of solution or preferably by one or more dry impregnations and very preferably by a single dry impregnation of said at least partially spent catalyst regenerated beforehand in stage a), using the impregnation solution.

The phosphoric acid and the organic acid can be introduced together in a single impregnation stage (coimpregnation) or independently in several impregnation stages, and this in any order.

Advantageously, after each impregnation stage, the impregnated regenerated catalyst is left to mature. Maturation makes it possible for the impregnation solution to disperse homogeneously within the regenerated catalyst.

Any maturation stage is advantageously carried out at atmospheric pressure, in a water-saturated atmosphere and at a temperature of between 17° C. and 50° C., and preferably at ambient temperature. Generally, a maturation time of between ten minutes and forty-eight hours, preferably of between thirty minutes and fifteen hours and particularly preferably between thirty minutes and six hours is sufficient.

When several impregnation stages are carried out, each impregnation stage is preferably followed by an intermediate drying stage at a temperature of less than 200° C., advantageously of between 50° C. and 180° C., preferably between 70° C. and 150° C., very preferably between 75° C. and 130° C., and optionally a maturation period has been observed between the impregnation stage and the intermediate drying stage.

### Drying (Step c))

After the rejuvenation stage, the catalyst is subjected to a drying stage at a temperature of less than 200° C., advantageously of between 50° C. and 180° C., preferably between 70° C. and 150° C., very preferably between 75° C. and 130° C., without a subsequent calcination stage.

The drying stage is preferentially carried out under an inert atmosphere or under an oxygen-containing atmosphere.

The drying stage can be carried out by any technique known to a person skilled in the art. It is advantageously carried out at atmospheric pressure or at reduced pressure. Preferably, this stage is carried out at atmospheric pressure. It is advantageously carried out in a traversed bed using hot air or any other hot gas. Preferably, when the drying is carried out in a fixed bed, the gas used is either air or an inert gas, such as argon or nitrogen. Very preferably, the drying is carried out in a traversed bed in the presence of nitrogen and/or air. Preferably, the drying stage has a duration of between 5 minutes and 4 hours, preferably between 30 minutes and 4 hours and very preferably between 1 hour and 3 hours.

The drying is carried out so as to preferably retain at least 30% by weight of the organic acid introduced during an impregnation stage; preferably, this amount is greater than 50% by weight and more preferably still greater than 70% by weight, calculated on the basis of the carbon remaining on the rejuvenated catalyst.

It is important to emphasize that the rejuvenated catalyst does not undergo calcination after the introduction of the phosphoric acid and of the organic acid in order to preserve, at least in part, the organic acid in the catalyst. Calcination is understood here to mean a heat treatment under a gas containing air or oxygen at a temperature of greater than or equal to 200° C.

On conclusion of the drying stage, a rejuvenated catalyst is then obtained, which catalyst will preferably be subjected to an optional activation (sulfidation) stage for its subsequent use in a hydrotreating and/or hydrocracking process.

### Rejuvenated Catalyst

The rejuvenated catalyst is composed of the oxide support not comprising zeolite and of the active phase formed of at least one metal from group VIb and of at least one metal from group VIII, of phosphorus and of the organic acid and contains a proportion of crystalline phase resulting from at least one metal from group VIII and from at least one metal from group VIb determined by X-ray diffraction and characterized by a ratio of the surface area of the diffraction peak of the crystal at 26.6° 2θ to the surface area of the peak characteristic of alumina at 45.7° 2θ of less than 0.4.

The total content of metal from group VIII is of between 1% and 15% by weight of oxide of the metal from group VIII, with respect to the total weight of the rejuvenated catalyst, preferably of between 1.5% and 12% by weight, preferably of between 2% and 10% by weight, of oxide of the metal from group VIII, with respect to the total weight of the rejuvenated catalyst.

The total content of metal from group VIb is of between 5% and 45% by weight of oxide of the metal from group VIb, with respect to the total weight of the rejuvenated catalyst, preferably of between 8% and 40% by weight, very preferably of between 10% and 30% by weight, of oxide of the metal from group VIb, with respect to the total weight of the rejuvenated catalyst.

The metal from group VIII to metal from group VIb molar ratio of the rejuvenated catalyst is generally of between 0.1 and 0.8, preferably of between 0.2 and 0.6.

The proportion of crystalline phase resulting from at least one metal from group VIII and from at least one metal from group VIb determined by X-ray diffraction and characterized by a ratio of the surface area of the diffraction peak of the crystal at 26.6° 2θ to the surface area of the peak characteristic of alumina at 45.7° 2θ is less than 0.4, preferably less than 0.35, in a preferred way less than 0.3 and even more preferably less than 0.25. The rejuvenated catalyst may also not contain crystalline phase.

The content of organic acid(s) on the rejuvenated catalyst is of between 1% and 45% by weight, preferably between 2% and 30% by weight and more preferably between 3% and 25% by weight, with respect to the total weight of the rejuvenated catalyst.

The total content of phosphorus (introduced by phosphoric acid during stage b) and optionally already present in the regenerated catalyst) in the rejuvenated catalyst is generally of between 0.3% and 25% by weight of P₂O₅, with respect to the total weight of catalyst, preferably between 0.5% and 20% by weight of P₂O₅, with respect to the total weight of catalyst, very preferably of between 1% and 15% by weight of P₂O₅, with respect to the total weight of catalyst.

### Sulfidation (Optional Stage)

Before its use in the hydrotreating and/or hydrocracking reaction, it is advantageous to transform the rejuvenated catalyst obtained according to the process according to the invention into a sulfided catalyst in order to form its active entity. This activation or sulfidation stage is carried out by methods well known to a person skilled in the art, and advantageously under a sulfo-reductive atmosphere in the presence of hydrogen and of hydrogen sulfide.

On conclusion of stage c) of the rejuvenation process according to the invention, said rejuvenated catalyst is thus advantageously subjected to a sulfidation stage, without an intermediate calcination stage.

Said rejuvenated catalyst is advantageously sulfided ex situ or in situ. The sulfiding agents are H₂S gas, elemental sulfur, CS₂, mercaptans, sulfides and/or polysulfides, hydrocarbon cuts having a boiling point of less than 400° C. containing sulfur-based compounds or any other sulfur-containing compound used for the activation of hydrocarbon feedstocks with a view to sulfiding the catalyst. Said sulfur-containing compounds are advantageously chosen from alkyl disulfides, such as, for example, dimethyl disulfide (DMDS), alkyl sulfides, such as, for example, dimethyl sulfide, thiols, such as, for example, n-butyl mercaptan (or 1-butanethiol), and polysulfide compounds of tert-nonyl polysulfide type. The catalyst can also be sulfided by the sulfur contained in the feedstock to be desulfurized. Preferably, the catalyst is sulfided in situ in the presence of a sulfiding agent and of a hydrocarbon feedstock. Very preferably, the catalyst is sulfided in situ in the presence of a hydrocarbon feedstock additivated with dimethyl disulfide.

Hydrotreating and/or Hydrocracking Process

Finally, another subject matter of the invention is the use of the rejuvenated catalyst according to the invention in processes for the hydrotreating and/or hydrocracking of hydrocarbon cuts.

The process for the hydrotreating and/or hydrocracking of hydrocarbon cuts can be carried out in one or more reactors in series of the fixed bed type or of the ebullated bed type.

The process for the hydrotreating and/or hydrocracking of hydrocarbon cuts is carried out in the presence of a rejuvenated catalyst. It can also be carried out in the presence of a mixture of a rejuvenated catalyst and of a fresh catalyst or of a regenerated catalyst.

When the fresh or regenerated catalyst is present, it comprises at least one metal from group VIII, at least one metal from group VIb and an oxide support, and optionally phosphorus and/or an organic compound such as are described above.

The active phase and the support of the fresh or regenerated catalyst may or may not be identical to the active phase and to the support of the rejuvenated catalyst.

The active phase and the support of the fresh catalyst may or may not be identical to the active phase and to the support of the regenerated catalyst.

When the process for the hydrotreating and/or hydrocracking of hydrocarbon cuts is carried out in the presence of a rejuvenated catalyst and of a fresh or regenerated catalyst, it can be carried out in a reactor of the fixed bed type containing several catalytic beds.

In this case, and according to a first alternative form, a catalytic bed containing the fresh or regenerated catalyst can precede a catalytic bed containing the rejuvenated catalyst in the direction of the circulation of the feedstock.

In this case, and according to a second alternative form, a catalytic bed containing the rejuvenated catalyst can precede a catalytic bed containing the fresh or regenerated catalyst in the direction of the circulation of the feedstock.

In this case, and according to a third alternative form, a catalytic bed can contain a mixture of a rejuvenated catalyst and of a fresh catalyst and/or of a rejuvenated catalyst.

In these cases, the operating conditions are those described above. They are generally identical in the different catalytic beds except for the temperature, which generally increases in a catalytic bed following the exothermicity of the hydrodesulfurization reactions.

When the process for the hydrotreating and/or hydrocracking of hydrocarbon cuts is carried out in the presence of a rejuvenated catalyst and of a fresh or regenerated catalyst in several reactors in series of the fixed bed type or of the ebullated bed type, one reactor can comprise a rejuvenated catalyst while another reactor can comprise a fresh or regenerated catalyst, or a mixture of a rejuvenated catalyst and of a fresh and/or regenerated catalyst, and this in any order. It is possible to provide a device for the removal of the H₂S from the effluent resulting from the first hydrodesulfurization reactor before treating said effluent in the second hydrodesulfurization reactor. In these cases, the operating conditions are those described above and may or may not be identical in the different reactors.

The rejuvenated catalyst which has preferably undergone a sulfidation stage beforehand is advantageously used in reactions for the hydrotreating and/or hydrocracking of hydrocarbon feedstocks, such as petroleum cuts, cuts resulting from coal, or hydrocarbons produced from natural gas, optionally as mixtures, or also from a hydrocarbon cut resulting from biomass and more in particularly reactions for the hydrogenation, hydrodenitrogenation, hydrodearomatization, hydrodesulfurization, hydrodeoxygenation, hydrodemetallization or hydroconversion of hydrocarbon feedstocks.

In these uses, the rejuvenated catalyst which has preferably undergone a sulfidation stage beforehand exhibits an improved activity in comparison with the catalysts of the prior art. This catalyst can also advantageously be used during the pretreatment of catalytic cracking or hydrocracking feedstocks, or the hydrodesulfurization of residues or the forced hydrodesulfurization of gas oils (ULSD: Ultra Low Sulfur Diesel).

The feedstocks employed in the hydrotreating process are, for example, gasolines, gas oils, vacuum gas oils, atmospheric residues, vacuum residues, atmospheric distillates, vacuum distillates, heavy fuel oils, oils, waxes and paraffins, spent oils, deasphalted residues or crudes, feedstocks originating from thermal or catalytic conversion processes, lignocellulose feedstocks or, more generally, feedstocks resulting from biomass, taken alone or as a mixture. The feedstocks which are treated, and in particular those mentioned above, generally contain heteroatoms, such as sulfur, oxygen and nitrogen, and, for the heavy feedstocks, they usually also contain metals.

The operating conditions used in the processes implementing the reactions for the hydrotreating of hydrocarbon feedstocks described above are generally as follows: the temperature is advantageously of between 180° C. and 450° C. and preferably between 250° C. and 440° C., the pressure is advantageously of between 0.5 and 30 MPa and preferably between 1 and 18 MPa, the hourly space velocity is advantageously of between 0.1 and 20 h⁻¹ and preferably between 0.2 and 5 h⁻¹, and the hydrogen/feedstock ratio, expressed as volume of hydrogen, measured under standard temperature and pressure conditions, per volume of liquid feedstock, is advantageously of between 50 I/I and 5000 I/I and preferably 80 and 2000 I/I.

According to a first mode of use, said hydrotreating process is a process for the hydrotreating, and in particular for the hydrodesulfurization (HDS), of a gas oil cut carried out in the presence of at least one rejuvenated catalyst according to the invention. Said hydrotreating process is targeted at removing the sulfur-based compounds present in said gas oil cut so as to meet the environmental standards in force, namely a permitted sulfur content of up to 10 ppm. It also makes it possible to reduce the contents of aromatics and of nitrogen of the gas oil cut to be hydrotreated.

Said gas oil cut to be hydrotreated contains from 0.02% to 5.0% by weight of sulfur. It advantageously results from straight-run distillation (or straight-run gas oil), from a coking unit, from a visbreaking unit, from a steam cracking unit, from a unit for the hydrotreating and/or hydrocracking of heavier feedstocks and/or from a catalytic cracking (Fluid Catalytic Cracking) unit. Said gas oil cut preferentially exhibits at least 90% of compounds with a boiling point of between 250° C. and 400° C. at atmospheric pressure.

The process for the hydrotreating of said gas oil cut is carried out under the following operating conditions: a temperature of between 200° C. and 400° C., preferentially between 300° C. and 380° C., a total pressure of between 2 MPa and 10 MPa and more preferentially between 3 MPa and 8 MPa, with a ratio of the volume of hydrogen per volume of hydrocarbon feedstock, expressed as volume of hydrogen, measured under standard temperature and pressure conditions, per volume of liquid feedstock, of between 100 and 600 liters per liter and more preferentially between 200 and 400 liters per liter, and an hourly space velocity (HSV) of between 1 and 10 h⁻¹, preferentially between 2 and 8 h⁻¹. The HSV corresponds to the inverse of the contact time expressed in hours and is defined by the ratio of the flow rate by volume of liquid hydrocarbon feedstock per volume of catalyst charged to the reaction unit implementing the hydrotreating process according to the invention. The reaction unit implementing the process for the hydrotreating of said gas oil cut is preferentially operated as a fixed bed, as a moving bed or as an ebullated bed, preferably as a fixed bed.

According to a second mode of use, said hydrotreating and/or hydrocracking process is a process for the hydrotreating (in particular hydrodesulfurization, hydrodenitrogenation, hydrogenation of aromatics) and/or hydrocracking of a vacuum distillate cut carried out in the presence of at least one rejuvenated catalyst according to the invention. Said hydrotreating and/or hydrocracking process, otherwise known as hydrocracking pretreatment or hydrocracking process, is targeted, as the case may be, at removing the sulfur-based, nitrogen-based or aromatic compounds present in said distillate cut so as to carry out a pretreatment before conversion in catalytic cracking or hydroconversion processes, or at hydrocracking the distillate cut which would optionally have been pretreated beforehand, if necessary.

Highly varied feedstocks can be treated by the processes for the hydrotreating and/or hydrocracking of vacuum distillates described above. Generally, they contain at least 20% by volume and often at least 80% by volume of compounds boiling above 340° C. at atmospheric pressure. The feedstock can, for example, be vacuum distillates and also feedstocks originating from units for the extraction of aromatics from lubricant oil bases or resulting from the solvent dewaxing of lubricant oil bases, and/or from deasphalted oils, or also the feedstock can be a deasphalted oil or paraffins resulting from the Fischer-Tropsch process, or also any mixture of the feedstocks mentioned above. In general, the feedstocks have a T5 boiling point of greater than 340° C. at atmospheric pressure and better still of greater than 370° C. at atmospheric pressure, that is to say that 95% of the compounds present in the feedstock have a boiling point of greater than 340° C. and better still of greater than 370° C. The nitrogen content of the feedstocks treated in the processes according to the invention is usually greater than 200 ppm by weight, preferably of between 500 and 10 000 ppm by weight. The sulfur content of the feedstocks treated in the processes according to the invention is usually of between 0.01% and 5.0% by weight. The feedstock can optionally contain metals (for example nickel and vanadium). The content of asphaltenes is generally less than 3000 ppm by weight.

The rejuvenated catalyst is generally brought into contact, in the presence of hydrogen, with the feedstocks described above, at a temperature of greater than 200° C., often of between 250° C. and 480° C., advantageously of between 320° C. and 450° C., preferably between 330° C. and 435° C., under a pressure of greater than 1 MPa, often of between 2 and 25 MPa, preferably between 3 and 20 MPa, the space velocity being of between 0.1 and 20.0 h⁻¹ and preferably 0.1-6.0 h⁻¹, preferably 0.2-3.0 h⁻¹, and the amount of hydrogen introduced is such that the liter of hydrogen/liter of hydrocarbon ratio by volume, expressed as volume of hydrogen, measured under standard temperature and pressure conditions, per volume of liquid feedstock, is of between 80 and 5000 I/I and most often between 100 and 2000 I/I. These operating conditions used in the processes according to the invention generally make it possible to obtain conversions per pass, into products having boiling points of less than 340° C. at atmospheric pressure and better still of less than 370° C. at atmospheric pressure, of greater than 15% and more preferably still of between 20% and 95%.

The processes for the hydrotreating and/or hydrocracking of vacuum distillates employing the rejuvenated catalysts according to the invention cover the pressure and conversion ranges extending from mild hydrocracking to high-pressure hydrocracking. Mild hydrocracking is understood to mean a hydrocracking which results in moderate conversions, generally of less than 40%, and which operates at low pressure, generally between 2 MPa and 6 MPa.

The rejuvenated catalyst according to the invention can be used alone, in a single or several fixed bed catalytic beds, in one or more reactors, in a “one-stage” hydrocracking scheme, with or without liquid recycling of the unconverted fraction, or also in a “two-stage” hydrocracking scheme, optionally in combination with a hydrorefining catalyst located upstream of the rejuvenated catalyst.

According to a third mode of use, said hydrotreating and/or hydrocracking process is advantageously employed as pretreatment in a fluidized bed catalytic cracking (or FCC: Fluid Catalytic Cracking) process. The operating conditions of the pretreatment in terms of range of temperature, pressure, hydrogen recycling rate and hourly space velocity are generally identical to those described above for the processes for the hydrotreating and/or hydrocracking of vacuum distillates. The FCC process can be carried out in a conventional way known to a person skilled in the art under suitable cracking conditions for the purpose of producing hydrocarbon products of lower molecular weight. A summary description of catalytic cracking will be found, for example, in Ullmann's Encyclopedia of Industrial Chemistry, Volume A18, 1991, pages 61 to 64.

According to a fourth mode of use, said hydrotreating and/or hydrocracking process according to the invention is a process for the hydrotreating (in particular hydrodesulfurization) of a gasoline cut in the presence of at least one rejuvenated catalyst according to the invention.

Unlike other hydrotreating processes, the hydrotreating (in particular the hydrodesulfurization) of gasolines has to make it possible to respond to a twofold conflicting constraint: to provide extreme hydrodesulfurization of gasolines and to limit the hydrogenation of the unsaturated compounds present in order to limit the loss in octane number.

The feedstock is generally a hydrocarbon cut having a distillation range of between 30° C. and 260° C. Preferably, this hydrocarbon cut is a cut of the gasoline type. Very preferably, the gasoline cut is an olefinic gasoline cut resulting, for example, from a catalytic cracking (Fluid Catalytic Cracking) unit.

The hydrotreating process consists in bringing the hydrocarbon cut into contact with the rejuvenated catalyst and hydrogen under the following conditions: at a temperature of between 200° C. and 400° C., preferably of between 230° C. and 330° C., at a total pressure of between 1 and 3 MPa, preferably of between 1.5 and 2.5 MPa, at an hourly space velocity (HSV), defined as being the flow rate by volume of feedstock with respect to the volume of catalyst, of between 1 and 10 h⁻¹, preferably of between 2 and 6 h⁻¹, and at a hydrogen/gasoline feedstock ratio by volume of between 100 and 600 SI/I, preferably of between 200 and 400 SI/I.

The process for the hydrotreating of the gasolines can be carried out in one or more reactors in series of the fixed bed type or of the ebullated bed type. If the process is carried out by means of at least two reactors in series, it is possible to provide a device for the removal of the H₂S from the effluent resulting from the first hydrodesulfurization reactor before treating said effluent in the second hydrodesulfurization reactor.

The examples which follow demonstrate the significant gain in activity on the rejuvenated catalysts prepared according to the process according to the invention in comparison with the catalysts of the prior art.

### EXAMPLES

### Example 1: Obtaining the Regenerated Catalyst A1

A hydrotreating catalyst was used in a refinery for 2 years on a unit for the hydrotreating of gas oil. The spent catalyst contains 10% by weight of carbon and 9% of sulfur. After a deoiling stage, the catalyst undergoes a regeneration under an oxidizing atmosphere at 480° C. The regenerated catalyst A1, which contains nickel, molybdenum and phosphorus, the contents of which as oxide equivalents are 4.5% NiO, 20.3% MoO₃ and 4.4% P₂O₅, supported on a gamma alumina, is obtained. The water retention volume of the catalyst A1 is 0.4 cc/g. This catalyst has carbon and sulfur contents respectively of 0.03% by weight and 0.2% by weight. Its ratio of the surface areas of the diffraction peaks NiMoO₄ (26.6° 2θ)/γ-Al₂O₃ (45.7° 2θ) is 0.85.

### Example 2: Obtaining the Regenerated Catalysts A2, A3, A4, A5, A6

The same spent and deoiled catalyst from example 1 undergoes regeneration under an oxidizing atmosphere at different temperatures: 450° C., 400° C., 380° C., 360° C. and 340° C. in order to respectively obtain the regenerated catalysts A2, A3, A4, A5 and A6 which have a water retention volume of 0.4 cc/g.

The catalyst A2 has carbon and sulfur contents respectively of 0.05% by weight and 0.3% by weight. Its ratio of the surface areas of the diffraction peaks NiMoO₄ (26.6° 2θ)/γ-Al₂O₃ (45.7° 2θ) is 0.78.

The catalyst A3 has carbon and sulfur contents respectively of 0.1% by weight and 0.6% by weight. Its ratio of the surface areas of the diffraction peaks NiMoO₄ (26.6° 2θ)/γ-Al₂O₃ (45.7° 2θ) is 0.47.

The catalyst A4 has carbon and sulfur contents respectively of 0.3% by weight and 0.7% by weight. Its ratio of the surface areas of the diffraction peaks NiMoO₄ (26.6° 2θ)/γ-Al₂O₃ (45.7° 2θ) is 0.23.

The catalyst A5 has carbon and sulfur contents respectively of 0.5% by weight and 0.8% by weight. Its ratio of the NiMoO₄/alumina areas is 0.11.

The catalyst A6 has carbon and sulfur contents respectively of 1.8% by weight and 1.4% by weight. Its ratio of the surface areas of the diffraction peaks NiMoO₄ (26.6° 2θ)/γ-Al₂O₃ (45.7° 2θ) is 0.10.

### Example 3: Obtaining the Catalysts B1 and B2 not in Accordance with the Invention

The catalyst B1 is prepared from the regenerated catalyst A1 on which a solution containing phosphoric acid and gluconic acid is dry impregnated, so as to obtain, on the rejuvenated catalyst, the P/Mo molar ratio of 0.8 and the gluconic acid/Mo molar ratio of 0.8. After a maturation of 3 h, the catalyst is dried at 120° C. for 2 h. Its ratio of the surface areas of the diffraction peaks NiMoO₄ (26.6° 2θ)/γ-Al₂O₃ (45.7° 2θ) is 0.57. The catalyst B2 is obtained with the same stages but starting from the regenerated catalyst A2. Its ratio of the surface areas of the diffraction peaks NiMoO₄ (26.6° 2θ)/γ-Al₂O₃ (45.7° 2θ) is 0.45.

### Example 4: Obtaining the Catalysts B3, B4 and B5 in Accordance with the Invention

The catalyst B3 is prepared from the regenerated catalyst A3 on which a solution containing phosphoric acid and gluconic acid is dry impregnated, so as to obtain, on the rejuvenated catalyst, the P/Mo molar ratio of 0.8 and the gluconic acid/Mo molar ratio of 0.8. After a maturation of 3 h, the catalyst is dried at 120° C. for 2 h. Its ratio of the surface areas of the diffraction peaks NiMoO₄ (26.6° 2θ)/γ-Al₂O₃ (45.7° 2θ) is 0.20. The catalysts B4 and B5 are obtained with the same stages but respectively starting from the regenerated catalysts A4 and A5. Their ratios of the surface areas of the diffraction peaks NiMoO₄ (26.6° 2θ)/γ-Al₂O₃ (45.7° 2θ) are respectively 0.13 and 0.06.

### Example 5: Obtaining the Catalyst B6 not in Accordance with the Invention

The catalyst B6 is prepared from the regenerated catalyst A6 on which a solution containing phosphoric acid and gluconic acid is dry impregnated, so as to obtain, on the rejuvenated catalyst, the P/Mo molar ratio of 0.8 and the gluconic acid/Mo molar ratio of 0.8. After a maturation of 3 h, the catalyst is dried at 120° C. for 2 h. Its ratio of the surface areas of the diffraction peaks NiMoO₄ (26.6° 2θ)/γ-Al₂O₃ (45.7° 2θ) is 0.05.

TABLE 1
Description of the catalysts and catalytic performance qualities in HOA of gas oil

| | | | | % by | % by | | | |
|---|---|---|---|---|---|---|---|---|
| | | | | weight | weight | Rege | Reju | |
| Regenerated | Rejuvenated | | | C | S | NiMo | NiMo | |
| catalyst | catalyst | | Rege | rege | rege | O₄/ | O₄/ | RVA |
| reference | reference | | T° C. | cata | cata | Al₂O₃* | Al₂O₃* | HOA |
| A1 | B1 | comparative | 480 | 0.03 | 0.2 | 0.85 | 0.57 | 100 |
| A2 | B2 | comparative | 450 | 0.05 | 0.3 | 0.78 | 0.45 | 106 |
| A3 | B3 | invention | 400 | 0.1 | 0.6 | 0.47 | 0.20 | 113 |
| A4 | B4 | invention | 380 | 0.3 | 0.7 | 0.23 | 0.13 | 114 |
| A5 | B5 | invention | 360 | 0.5 | 0.8 | 0.11 | 0.06 | 118 |
| A6 | B6 | comparative | 340 | 1.8 | 1.4 | 0.10 | 0.05 | 107 |

*ratio of the surface areas of the diffraction peaks NiMoO₄ (26.6° 2θ)/γ-Al₂O₃ (45.7° 2θ)

### Example 6: Evaluation in Hydrogenation of the Aromatics (HOA) of Gas Oil of the Catalysts B1, B2 and B6 (not in Accordance with the Invention) and B3, B4 and B5 (in Accordance with the Invention)

The catalysts B1, B2 and B6 (not in accordance with the invention) and B3, B4 and B5 (in accordance with the invention) were tested in HOA of gas oil. The regenerated catalyst B1 serves as reference.

The feedstock is a mixture of 30% by volume of gas oil resulting from atmospheric distillation (also referred to as straight-run distillation) and 70% by volume of light gas oil resulting from a catalytic cracking unit (also known as LCO for Light Cycle Oil). The characteristics of the test feedstock used are as follows: density at 15° C.=0.8994 g/cm³ (NF EN ISO 12185), refractive index at 20° C.=1.5143 (ASTM D1218-12), sulfur content=0.38% by weight, nitrogen content=0.05% by weight.
Simulated distillation (ASTM D2887):
IP: 133° C.
10%: 223° C.
50%: 285° C.
90%: 357° C.
FP: 419° C.

The test is carried out in an isothermal pilot reactor having a traversed fixed bed, the fluids circulating from the bottom upward.

The catalysts are sulfided beforehand in situ at 350° C. in the reactor under pressure by means of an atmospheric (straight-run) distillation gas oil feedstock (density at 15° C.=0.8491 g/cm³ (NF EN ISO 12185) and initial sulfur content=0.42% by weight), to which 2% by weight of dimethyl disulfide are added.

The tests of hydrogenation of the aromatics were carried out under the following operating conditions: a total pressure of 8 MPa, a catalyst volume of 4 cm³, a temperature of 330° C., with a hydrogen flow rate of 3.0 l/h and with a feedstock flow rate of 4.5 cm³/h.

The characteristics of the effluents are analyzed: density at 15° C. (NF EN ISO 12185), refractive index at 20° C. (ASTM D1218-12), simulated distillation (ASTM D2887), sulfur content and nitrogen content. The residual contents of aromatic carbon are calculated by the n-d-M method (ASTM D3238). The degree of hydrogenation of the aromatics is calculated as the ratio of the content of aromatic carbon of the effluents to that of the test feedstock. The catalytic performance qualities of the catalysts tested are given in table 1. They are expressed as relative volume activity (RVA) with respect to the catalyst B1 chosen as reference, assuming an order of 1.7 for the reaction concerned.

The catalysts B3, B4 and B5 in accordance with the invention exhibit the best activities beyond RVA 110 because they were prepared on regenerated catalysts exhibiting simultaneously carbon and sulfur contents respectively of between 0.1% and 0.5% by weight and 0.3% and 0.8% by weight S and a ratio of the surface areas of the diffraction peaks NiMoO₄ (26.6° 2θ)/γ—Al₂O₃ (45.7° 2θ) of less than 0.6 by virtue of controlled regeneration. The rejuvenation stage 10 subsequently makes it possible to dissolve, in part, the NiMoO₄ crystalline phase in order to redisperse the molybdenum- and nickel-based entities and to thus obtain rejuvenated catalysts according to the invention exhibiting a ratio of the surface areas of the diffraction peaks NiMoO₄ (26.6° 2θ)/γ-Al₂O₃ (45.7° 2θ) of less than 0.4.

1. A process for the rejuvenation of an at least partially spent catalyst resulting from a hydrotreating and/or hydrocracking process, said at least partially spent catalyst resulting from a fresh catalyst comprising at least one metal from group VIII, at least one metal from group VIb, an oxide support not comprising zeolite, and optionally phosphorus, said process comprises the following stages:
a) the at least partially spent catalyst is regenerated in an oxygen-containing gas stream at a temperature of between 360° C. and less than 420° C. so as to obtain a regenerated catalyst comprising a carbon content of between 0.1% and 0.5% by weight, a sulfur content of between 0.3% and 0.8% by weight and a proportion of crystalline phase resulting from at least one metal from group VIII and from at least one metal from group VIb determined by X-ray diffraction and characterized by a ratio of the surface area of the diffraction peak of the crystal at 26.6° 2θ to the surface area of the peak characteristic of alumina at 45.7° 2θ of less than 0.6,
b) then said regenerated catalyst is brought into contact with an aqueous solution consisting of water, of phosphoric acid and of an organic acid having each acidity constant pKₐ greater than 1.5,
c) a drying stage is carried out at a temperature of less than 200° C., without subsequently calcining it, so as to obtain a rejuvenated catalyst.

2. The process as claimed in claim 1, in which the temperature of stage a) is of between 380° C. and 410° C.

3. The process as claimed in claim 1, in which the proportion of crystalline phase resulting from at least one metal from group VIII and from at least one metal from group VIb determined by X-ray diffraction and characterized by a ratio of the surface area of the diffraction peak of the crystal at 26.6° 2θ to the surface area of the peak characteristic of alumina at 45.7° 2θ in stage a) is less than 0.50.

4. The process as claimed in claim 1s, in which the organic acid used in stage b) is chosen from gluconic acid, tartaric acid, citric acid, γ-ketovaleric acid, lactic acid, pyruvic acid, ascorbic acid or succinic acid.

5. The process as claimed in claim 1, in which the organic acid used in stage b) is an organic acid having each acidity constant pKₐ greater than 3.5.

6. The process as claimed in claim 1, in which the organic acid used in stage b) is chosen from gluconic acid, γ-ketovaleric acid, lactic acid, pyruvic acid, ascorbic acid or succinic acid.

7. The process as claimed in claim 1, in which the organic acid added per metal/metals from group VIb present in the regenerated catalyst molar ratio is between 0.01 and 5 mol/mol.

8. The process as claimed in claim 1, in which the phosphorus added per metal from group VIb already present in the regenerated catalyst molar ratio is between 0.01 and 5 mol/mol.

9. The process as claimed in claim 1, in which the fresh catalyst has a content of metal from group VIb of between 1% and 40% by weight of oxide of said metal from group VIb, with respect to the weight of the catalyst, and a total content of metal from group VIII of between 1% and 10% by weight of oxide of said metal from group VIII, with respect to the weight of the catalyst.

10. The process as claimed in claim 1, in which the fresh catalyst contains phosphorus, the total content of phosphorus being of between 0.1% and 20% by weight, expressed as P₂O₅, with respect to the total weight of the catalyst.

11. The process as claimed in claim 1, in which the oxide support not comprising zeolite is chosen from aluminas, silica, silica-aluminas or also titanium or magnesium oxides, used alone or as a mixture with alumina or silica-alumina.

12. The process as claimed in claim 1, in which the rejuvenated catalyst resulting from stage c) contains a proportion of crystalline phase resulting from at least one metal from group VIII and from at least one metal from group VIb determined by X-ray diffraction and characterized by a ratio of the surface area of the diffraction peak of the crystal at 26.6° 2θ to the surface area of the peak characteristic of alumina at 45.7° 2θ of less than 0.4.

13. The process as claimed in claim 1, in which the regeneration stage a) is preceded by a deoiling stage which comprises bringing an at least partially spent catalyst resulting from a hydrotreating and/or hydrocracking process into contact with a stream of inert gas at a temperature of between 300° C. and 400° C.

14. The process as claimed in claim 1, in which the rejuvenated catalyst is subjected to a sulfidation stage after stage c).

15. A process for the hydrotreating and/or hydrocracking of hydrocarbon cuts comprising performing said hydrotreating and/or hydrocracking in the presence of the catalyst obtained according to the process as claimed in claim 1.
